(19) United States
(12) Patent Application Publication
(10) Pub. No.: US 2005/0163731 A1
(43) Pub. Date: Jul. 28, 2005

(54) Skin depigmenting compositions comprising adapalene and at least one depigmenting active agent

(75) Inventors: Isabelle Pelisson, Vallauris (FR); Andre Jomard, Saint Vallier De Thiey (FR)

Correspondence Address: BURNS DOANE SWECKER & MATHIS L L P, POST OFFICE BOX 1404, ALEXANDRIA, VA, 22313-1404, US

(73) Assignee: GALDERMA RESEARCH & DEVELOPMENT, S.N.C., Valbonne (FR)

(21) Appl. No.: 11/042,519

(22) Filed: Jan. 26, 2005

Related U.S. Application Data
(63) Continuation of application No. PCT/EP03/10692, filed on Sep. 1, 2003.
(60) Provisional application No. 60/411,350, filed on Sep. 18, 2002.

(30) Foreign Application Priority Data
Sep. 1, 2003 (WO) PCT/EP03/10692
Sep. 5, 2002 (FR) 02/11022

(51) Int. Cl. A61K 7/42; A61K 7/135

(52) U.S. Cl. 424/59; 424/62

(57) ABSTRACT

Topically applicable skin depigmenting compositions, suited for treatment of such hyperpigmentary disorders as melasma and chloasma, contain a thus effective amount of at least one skin depigmenting active agent, for example hydroquinone or 4-hydroxyanisole, in admixture with a depigmentation-accelerating amount of adapalene (6-[3-(1-adamantyl)-4-methoxyphenyl]-2-napthanoic acid), and optionally with at least one sunscreen, formulated into a topically applicable, physiologically acceptable medium therefor.

### CROSS-REFERENCE TO PRIORITY/PCT/PROVISIONAL APPLICATIONS

This application claims priority under 35 U.S.C. § 119 of FR-02/11022, filed Sep. 5, 2002, and of provisional application Ser. No. 60/411,350, filed Sep. 18, 2002, and is a continuation of PCT/EP 2003/010692, filed Sep. 1, 2003 and designating the United States (published in the English language on Mar. 18, 2004 as WO 2004/021967 A2), each hereby expressly incorporated by reference and each assigned to the assignee hereof.

### BACKGROUND OF THE INVENTION

1. Technical Field of the Invention

The present invention relates to depigmenting compositions for the skin comprising, formulated into a physiologically acceptable medium, adapalene (6-[3-(1-adamantyl)-4-methoxyphenyl]-2-naphthanoic acid) and at least one depigmenting active agent and to certain pharmaceutical and cosmetic applications thereof.

The subject compositions according to the invention may also contain a sunscreen.

2. Description of Background and/or Related and/or Prior Art

A. M. Kligman describes, in U.S. Pat. No. 3,856,934, a depigmenting composition comprising hydroquinone, retinoic acid and a corticosteroid. This type of combination allows good depigmentation of the skin but causes substantial undesirable effects such as irritation and itching.

Retinoic acid (tretinoin) is known to itself have a skin depigmenting activity (Guevara I. A. and Pandya A. G., Int. J. Dermatol., 40, 210-215 (2001)) unlike adapalene which has no depigmenting activity as is shown in Example 6 below. By virtue of its lack of depigmenting activity in particular, nothing therefore would motivate one skilled in this art to combine it with a depigmenting agent in a depigmenting composition, whether or not containing a sunscreen.

### SUMMARY OF THE INVENTION

It has now surprisingly and unexpectedly been determined that the combination or intimate admixture of adapalene and a depigmenting agent elicits a much more rapid depigmenting response than that obtained with the depigmenting agent alone.

The present invention therefore features depigmenting compositions for the skin comprising, in a physiologically acceptable medium, adapalene and at least one depigmenting agent.

The expression physiologically acceptable medium is understood to mean a medium compatible with the skin, the mucous membranes and/or the superficial body growths.

The expression depigmenting agent is understood to mean any biologically active agent having a skin depigmenting activity. This activity makes it possible to reduce the already existing pigmentation of the skin and also to prevent any additional pigmentation above the natural pigmentation.

### DETAILED DESCRIPTION OF BEST MODE AND SPECIFIC/PREFERRED EMBODIMENTS OF THE INVENTION

There may be mentioned, as representative depigmenting agents, by way of non-limiting examples, phenolic derivatives, such as hydroquinone, hydroquinone monoethyl ether, hydroquinone monobenzyl ether or 4-hydroxyanisole; kojic acid and its derivatives; azelaic acid and its derivatives; linoleic acid; resorcinol and its derivatives; ellagic acid; hydroxy acids such as glycolic acid; ascorbic acid and its derivatives, in particular ascorbyl glucoside; zinc peroxide; and mercury chloride, arbutin and its derivatives such as those described in applications EP-895,779 and EP-524,109; aminophenol derivatives such as those described in applications WO 99/10318 and WO 99/32077, and in particular N-cholesteryloxycarbonyl-para-aminophenol and N-ethyloxycarbonyl-para-aminophenol; iminophenol derivatives, in particular those described in application WO 99/22707; L-2-oxothiazolidine-4-carboxylic acid or procysteine, and its salts and esters; and extracts of plants, in particular of liquorice, mulberry and skullcap.

In particular, the depigmenting compositions for the skin can comprise, as depigmenting agent, a phenolic derivative, in particular hydroquinone or 4-hydroxyanisole.

This invention also features depigmenting compositions for the skin comprising, in a physiologically acceptable medium, adapalene, at least one depigmenting agent and at least one sunscreen.

To provide an order of magnitude, the compositions according to the invention advantageously comprise from 0.0001% to 20% by weight of adapalene relative to the total weight of the composition and from 0.0001% to 20% by weight of depigmenting agent relative to the total weight of the composition, and preferably, respectively, from 0.001% to 10% by weight of adapalene relative to the total weight of the composition and from 0.025% to 10% by weight of depigmenting agent relative to the total weight of the composition.

The subject compositions may also comprise at least one sunscreen in preferential concentrations ranging from 0.001 to 30.00% by weight relative to the total weight of the composition.

Among the sunscreens, there may be mentioned, by way of non-limiting example, physical sunscreens such as titanium dioxide, zinc oxide, and chemical sunscreens such as octocrylene, ethylhexyl methoxycinnamate, octyl salicylate, avobenzone, oxybenzone, ecamsule or drometrizole trisiloxane or mixtures thereof. Each sunscreen may be added at a concentration ranging from 0.001 to 20% by weight relative to the total weight of the composition.

The compositions according to the invention may additionally comprise any additive customarily used in the cosmetic or pharmaceutical field, such as sequestrants, antioxidants, preservatives, fillers, electrolytes, humectants, colorants, customary inorganic or organic bases or acids, perfumes, essential oils, cosmetic active agents, moisturizers, vitamins, essential fatty acids, sphingolipids, self-tanning compounds, skin soothing and protecting agents such as allantoin. Of course, one skilled in the art will be careful to choose this or these optional additional compounds, and/or their quantity, such that the advantageous properties of the composition according to the invention are not, or not substantially, impaired.

These additives may be present in the composition in an amount of 0.001 to 20% by weight relative to the total weight of the composition.

There may be mentioned as example of sequestering agents: ethylenediaminetetraacetic acid (EDTA), and its derivatives or its salts, dihydroxyethylglycine, citric acid and tartaric acid.

There may be mentioned as examples of preservatives: benzalkonium chloride, phenoxyethanol, benzyl alcohol, diazolidinylurea and parabens.

There may be mentioned as examples of humectants: glycerin and sorbitol.

The present invention also features regime or regimen for the administration of the subject compositions as medicaments.

This invention also features regime or regimen for the administration of the subject compositions for pharmaceutical and cosmetic applications.

The compositions of the invention are particularly suitable for the treatment and prevention of hyperpigmentary disorders such as melasma, chloasma, lentigines, senile lentigo, vitiligo, freckles, post-inflammatory hyperpigmentations due to an abrasion, a burn, a scar, a dermatosis, a contact allergy; naevi, hyperpigmentations with a genetic determinism, hyperpigmentations of metabolic or drug origin, melanomas or any other hyperpigmentary lesions.

The compositions according to the invention also find application in the cosmetic field, in particular for protection against the harmful effects of the sun, for preventing and/or combating photoinduced or chronologic aging of the skin and of the superficial body growths.

The present invention also features a regime or regimen comprising a non-therapeutic cosmetic treatment for beautifying the skin and/or for improving its surface appearance, wherein a composition comprising adapalene and at least one depigmenting agent is topically applied onto the skin and/or its superficial body growths.

In order to further illustrate the present invention and the advantages thereof, the following specific examples are given, it being understood that same are intended only as illustrative and in nowise limitative. In said examples to follow, all parts and percentages are given by weight, unless otherwise indicated.

Examples illustrating the depigmenting activity of the various compositions according to the invention are also described.

### EXAMPLE 1

| | |
|---|---|
| Adapalene | 0.1 |
| 4-Hydroxyanisole | 4 |
| Steareth-2 | 3 |
| Steareth-21 | 2 |
| Caprylic-capric triglyceride | 4 |
| Isohexadecane | 5 |
| Cetearyl alcohol | 1 |
| Stearic acid | 1.5 |
| Sodium sulphite | 0.2 |
| Propylene glycol | 8 |
| Glycerin | 2 |
| Phenoxyethanol | 1 |
| Citric acid (qs pH 5.5-6.0) | / |
| Purified water | qs 100 |

This composition should be applied twice per day until complete depigmentation is obtained for the treatment of lentigines.

### EXAMPLE 2

| | |
|---|---|
| Adapalene | 0.1 |
| Hydroquinone | 2 |
| Carbomer | 0.1 |
| Methyl Paraben | 0.18 |
| Propyl Paraben | 0.02 |
| Cetyl alcohol | 1 |
| Stearic acid | 0.8 |
| Caprylic-capric triglyceride | 1.5 |
| Cyclomethicone | 1 |
| Inorganic oil | 2 |
| Propylene glycol | 5 |
| Glycerin | 2 |
| Triethanolamine | 5 |
| Citric acid (qs pH 5.5-6.0) | / |
| Purified water | Qs 100 |

This composition should be applied once per day until complete depigmentation is obtained for the treatment of melasma.

### EXAMPLE 3

| | |
|---|---|
| Adapalene | 0.10 |
| Hydroquinone | 4.00 |
| Magnesium and aluminum silicate | 3.00 |
| Butylated hydroxytoluene | 0.04 |
| Methyl Paraben | 0.18 |
| Propyl Paraben | 0.02 |
| Cetyl alcohol | 4.00 |
| Stearic acid | 3.00 |
| Stearyl alcohol | 4.00 |
| Glyceryl Stearate/PEG-100 stearate | 3.50 |
| Methyl Gluceth-10 | 5.00 |
| Glycerin | 4.00 |
| Citric acid | 0.05 |
| Sodium metabisulphite | 0.20 |
| Purified water | qs 100 |

This composition should be applied twice per day until complete depigmentation is obtained for the treatment of melasma.

### EXAMPLE 4

| | |
|---|---|
| Adapalene | 0.10 |
| Hydroquinone | 4.00 |
| Magnesium and aluminum silicate | 3.00 |
| Butylated hydroxytoluene | 0.04 |
| Methyl Paraben | 0.18 |
| Propyl Paraben | 0.02 |
| Cetyl alcohol | 4.00 |
| Stearic acid | 3.00 |
| Stearyl alcohol | 4.00 |
| Glyceryl Stearate/PEG-100 stearate | 3.50 |
| Avobenzone | 2.00 |
| Titanium dioxide | 2.00 |
| Methyl Gluceth-10 | 5.00 |
| Glycerin | 4.00 |
| Citric acid | 0.05 |
| Sodium metabisulphite | 0.20 |
| Purified water | qs 100 |

This composition should be applied twice per day until complete depigmentation is obtained for the treatment of melasma.

### EXAMPLE 5

| | |
|---|---|
| Adapalene | 0.10 |
| Hydroquinone | 4.00 |
| Magnesium and aluminum silicate | 3.00 |
| Butylated hydroxytoluene | 0.04 |
| Methyl Paraben | 0.18 |
| Propyl Paraben | 0.02 |
| Cetyl alcohol | 4.00 |
| Stearic acid | 3.00 |
| Stearyl alcohol | 4.00 |
| Glyceryl Stearate/PEG-100 stearate | 3.50 |
| Ethylhexyl methoxycinnamate | 2.00 |
| Methyl Gluceth-10 | 5.00 |
| Glycerin | 4.00 |
| Citric acid | 0.05 |
| Sodium metabisulphite | 0.20 |
| Ecamsule | 3.00 |
| Purified water | qs 100 |

This composition should be applied once per day until complete depigmentation is obtained for the treatment of chloasma.

### EXAMPLE 6

### Measurement of the Depigmenting Activity of the Combination of Adapalene and a Depigmenting Agent

The evaluation of the depigmenting and/or anti-pigmenting activity of hydroquinone 3% and Adapalene 0.1% alone or in combination for 8 weeks is carried out on the tail of SKH HR2 mice which is irradiated or not with ultraviolet B. The tail of the SKH:HR2 mouse is naturally pigmented and this pigmentation increases under the effect of repeated UV-B irradiation. The depigmenting activity is measured on the natural pigmentation after application of the test product to the tail of non-irradiated animals.

The anti-pigmenting activity is measured by the inhibition of the induction of the UV-induced pigmentation: the test product is applied to the tail of animals irradiated with UV-B.

The treatment is carried out for 5 days per week for 8 weeks. 20 μl of test product, diluted in acetone, are applied to the tail in a deferred manner: hydroquinone in the morning and adapalene 4 h later. On the days for irradiation, the treatment is applied after irradiation.

The animals are irradiated 3 times per week for 8 weeks (Monday, Wednesday, Friday) at the dose of 90 mJ/cm² of UV-B.

The evaluation is made by different clinical observations: once per week before irradiation, the pigmentation is evaluated by virtue of a score on a scale from 0 to 4. The distribution of the scores is the following:
−0: natural pigmentation
depigmentation scale: scores −1 to −4
−1: light depigmentation
−2: moderate depigmentation
−3: marked depigmentation
−4: total depigmentation
pigmentation scale: scores 1 to 4
1: light pigmentation
2: moderate pigmentation
3: marked pigmentation
4: intense pigmentation

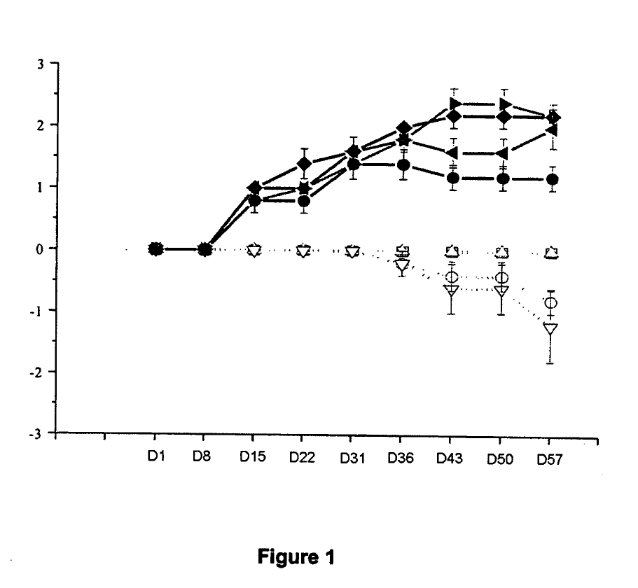

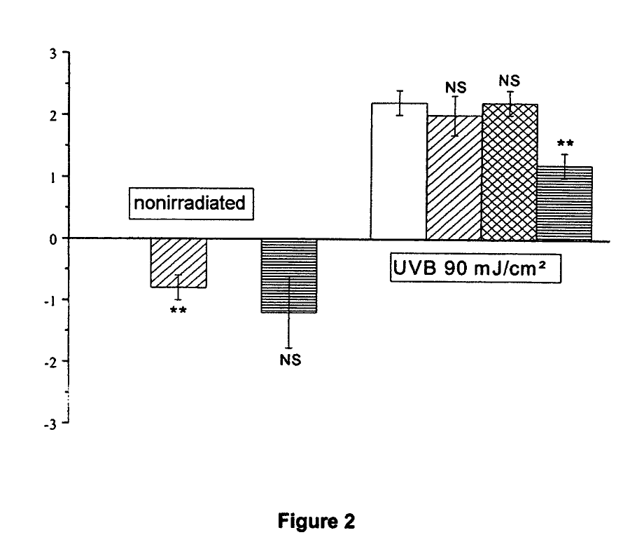

The results are shown in FIGS. 1 and 2.

FIG. 1 illustrates the kinetics for the scores of pigmentation of the mouse skin as a function of the treatment time with or without irradiation with ultraviolet B (up to 8 weeks of treatment) with (♦) UV-B+acetone, ( [symbol] ) UV-B+Adapalene, ( [symbol] ) UV-B+hydroquinone, (●) UV-B+hydroquinone+adapalene, ( [symbol] ) non-irradiated skin+acetone, (Δ) non-irradiated skin+adapalene (∘) non-irradiated skin+hydroquinone (∇) non-irradiated skin+hydroquinone+adapalene.

FIG. 2 illustrates the pigmentation scores at the end of the study (D57) with ( [symbol] ) acetone, ( [symbol] ) hydroquinone, ( [symbol] ) adapalene, ( [symbol] ) adapalene+hydroquinone with or without ultraviolet B irradiation.

Depigmenting activity: hydroquinone alone at 3% induces a clinically visible depigmentation from the 6th week of treatment (D43) and a statistically significant depigmentation at the end of the study. Adapalene alone does not modify the natural pigmentation. When adapalene is combined with Hydroquinone, it potentiates its depigmenting activity.

Anti-pigmenting activity: in the animals irradiated and treated concomitantly, hydroquinone shows an anti-pigmenting effect at the 6th and at the 7th week (D50) which becomes less marked at the end of the study. On the other hand, the adapalene+hydroquinone combination inhibits the pigmentation induced by UV-B irradiation from its appearance and up to the end of the study where the difference is statistically significant (**, p<0.01).

### CONCLUSION

After 8 weeks of topical treatment on the tail of SKH:HR2 mice, it is noted that the hydroquinone+adapalene combination exhibits a high anti-pigmenting activity and significantly inhibits the pigmentation induced by UV-B irradiation from its appearance and up to the end of the study. The hydroquinone+adapalene combination has a significant benefit as depigmenting agent in relation to hydroquinone alone.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A topically applicable skin depigmenting composition, comprising a thus effective amount of at least one skin depigmenting active agent, in admixture with a depigmentation-accelerating amount of adapalene (6-[3-(1-adamantyl)-4-methoxyphenyl]-2-napthanoic acid), formulated into a topically applicable, physiologically acceptable medium therefor.

2. The skin depigmenting composition as defined by claim 1, said at least one skin depigmenting active agent comprising a phenolic compound, hydroquinone, hydroquinone monoethyl ether, hydroquinone monobenzyl ether, 4-hydroxyanisole, kojic acid or derivative thereof, azelaic acid or derivative thereof, linoleic acid, resorcinol or derivative thereof, ellagic acid, a hydroxy acid, glycolic acid, ascorbic acid or derivative thereof, ascorbyl glucoside, zinc peroxide, mercury chloride, arbutin or derivative thereof, an aminophenol compound, N-cholesteryloxy-carbonyl-para-aminophenol, N-ethyloxycarbonyl-para-aminophenol, an iminophenol compound, L-2-oxothiazolidine-4-carboxylic acid or procysteine, or salt or ester thereof, a plant extract, a liquorice, mulberry or skullcap extract, or mixture thereof.

3. The skin depigmenting composition as defined by claim 2, said at least one skin depigmenting active agent comprising a phenolic compound.

4. The skin depigmenting composition as defined by claim 3, said at least one skin depigmenting active agent comprising hydroquinone or 4-hydroxyanisole.

5. The skin depigmenting composition as defined by claim 1, further comprising at least one sunscreen.

6. The skin depigmenting composition as defined by claim 5, said at least one sunscreen comprising titanium dioxide, zinc oxide, octocrylene, ethylhexyl methoxycinnamate, octyl salicylate, avobenzone, oxybenzone, ecamsule, drometrizole trisiloxane, or mixture thereof.

7. The skin depigmenting composition as defined by claim 1, further comprising at least one cosmetic/pharmaceutical additive.

8. The skin depigmenting composition as defined by claim 7, said at least one cosmetic/pharmaceutical additive comprising a sequestrant, antioxidant, preservative, filler, electrolyte, humectant, colorant, inorganic or organic base or acid, perfume, essential oil, cosmetic active agent, moisturizer, vitamin, essential fatty acid, sphingolipid, self-tanning compound, skin soothing and protecting agent, allantoin, or mixture thereof.

9. A topically applicable skin depigmenting composition, consisting essentially of a thus effective amount of at least one skin depigmenting active agent, in admixture with a depigmentation-accelerating amount of adapalene (6-[3-(1-adamantyl)-4-methoxyphenyl]-2-napthanoic acid), formulated into a topically applicable, physiologically acceptable medium therefor.

10. The skin depigmenting composition as defined by claim 1, comprising from 0.0001% to 20% by weight of adapalene.

11. The skin depigmenting composition as defined by claim 10, comprising from 0.0001% to 20% by weight of said at least one skin depigmenting active agent.

12. The skin depigmenting composition as defined by claim 10, comprising from 0.001% to 10% by weight of adapalene.

13. The skin depigmenting composition as defined by claim 12, comprising from 0.025% to 10% by weight of said at least one skin depigmenting active agent.

14. The skin depigmenting composition as defined by claim 5, each such sunscreen comprising from 0.001% to 30% by weight thereof.

15. A regime or regimen for the treatment of a pigmentary disorder of the skin, comprising topically applying onto the affected area of the skin of an individual in need of such treatment, for such period of time as required to elicit the desired response, a thus effective amount of at least one skin depigmenting active agent, in admixture with a depigmentation-accelerating amount of adapalene (6-[3-(1-adamantyl)-4-methoxyphenyl]-2-napthanoic acid), formulated into a topically applicable, physiologically acceptable medium therefor.

16. A regime or regimen for the prevention or treatment of a hyperpigmentary disorder of the skin, comprising topically applying onto the affected area of the skin of an individual in need of such treatment, for such period of time as required to elicit the desired response, a thus effective amount of the skin depigmenting composition as defined by claim 1.

17. The regime or regimen as defined by claim 16, for the prevention or treatment of melasma, chloasma, lentigines, vitiligo, freckles, post-inflammatory hyperpigmentation due to an abrasion, a burn, a scar, a dermatosis, a contact allergy, naevi, hyperpigmentation having a genetic determinism, hyperpigmentation of metabolic or drug origin, melanoma, or other hyperpigmentary lesion.

18. The regime or regimen as defined by claim 17, for the treatment of melasma.

19. The regime or regimen as defined by claim 17, for the treatment of chloasma.

20. A regime or regimen for photoprotecting the skin and superficial body growths and/or preventing and/or combating photoinduced or chronological aging thereof, comprising topically applying thereon a thus effective amount of the skin depigmenting composition as defined by claim 1.

21. A regime or regimen for photoprotecting the skin and superficial body growths and/or preventing and/or combating photoinduced or chronological aging thereof, comprising topically applying thereon a thus effective amount of the skin depigmenting composition as defined by claim 5.

22. A non-therapeutic cosmetic regime or regimen for beautifying the skin and superficial body growths and/or for improving the surface appearance thereof, comprising topically applying thereon a thus effective amount of the skin depigmenting composition as defined by claim 1.
